(19) United States
(12) Patent Application Publication
(10) Pub. No.: US 2007/0026430 A1
(43) Pub. Date: Feb. 1, 2007

(54) PROXIMITY PROBING OF TARGET PROTEINS COMPRISING RESTRICTION AND/OR EXTENSION

(75) Inventors: Mark R. Andersen, Carlsbad, CA (US); Benjamin G. Schroeder, San Mateo, CA (US); Kai Qin Lao, Pleasanton, CA (US)

Correspondence Address: MILA KASAN, PATENT DEPT.;APPLIED BIOSYSTEMS, 850 LINCOLN CENTRE DRIVE, FOSTER CITY, CA, 94404, US

(73) Assignee: APPLERA CORPORATION, Foster City, CA (US)

(21) Appl. No.: 11/428,191

(22) Filed: Jun. 30, 2006

Related U.S. Application Data
(60) Provisional application No. 60/696,108, filed on Jun. 30, 2005.

(51) Int. Cl. C12Q 1/68; C07H 21/04; C12P 19/34

(52) U.S. Cl. 435/6; 435/91.2; 536/24.3

(57) ABSTRACT

The present teachings provide methods, compositions, and kits for detecting target analytes, including proteins. In some embodiments, cleavage reactions are performed in the context of proximity probe reactions that query target proteins, wherein the presence and/or quantity of cleavage products is indicative of the presence and/or quantity of a target protein. In some embodiments, the cleavage fragments are quantitated using a real time PCR assay comprising a stem-loop primer, wherein the stem-loop primer comprises a self-complementary hairpin structure and a free 3′ end end complementary to the cleavage product. In some embodiments, polymerase extension approaches are employed in the context of proximity probe reactions.

### CROSS-REFERENCE TO RELATED APPLICATIONS

This application claims a priority benefit under 35 U.S.C. §119(e) from U.S. Patent Application No. 60/696,108, filed Jun. 30, 2005, the entire contents of which is incorporated herein by reference.

### FIELD

The present teachings relate to methods, compositions, and kits for detecting and/or quantitating analytes such as proteins in cleavage reactions, and extension reactions, comprising proximity probes bearing coupled nucleic acids.

### INTRODUCTION

Various forms of PCR are widely used to quantify specific nucleic acids targets. As proteomics gains momentum, there is an increasing need for simple assays to quantify protein concentration with high levels of sensitivity and specificity. Illustrative background teachings discussing method of detecting and quantitating proteins using nucleic acid amplification procedures can be found for example in Zhang et al., 2001, PNAS 98 (10): 5497-5502, Fredriksson et al., (2003) Nature Biotechnology 20:473-7, and Published PCT Application WO 03/044231A1, Sano et al., U.S. Pat. No. 5,665,539, Baez et al., U.S. Pat. No. 6,511,809, and Feaver et al., U.S. patent application Ser. No. 10/454,946.

The development of immunoassays and advances in methods of nucleic acid amplification have significantly advanced the art of the detection of biological analytes. In spite of these advances, nonspecific binding of the analyte to be detected and general assay noise has remained a problem that has limited the application and sensitivity of such assays. Methods for the reduction of background noise are continually being sought.

The introduction of immunoassays in the 1960's and 1970's greatly increased the number of analytes amenable to precise and accurate measurement. Radio-immunoassays (RIAs) and immunoradiometric (IRMA) assays utilize radioisotopic labeling of either an antibody or a competing analyte to measure an analyte. Detection systems based on enzymes or fluorescent labels were then developed as an alternative to isotopic detection systems. D. L. Bates, Trends in Biotechnology, 5(7), 204 (1987), describes one such method based upon enzyme amplification. In this method a secondary enzyme system is coupled to a primary enzyme label. For example, the primary enzyme can be linked catalytically to an additional system such as a substrate cycle or an enzyme cascade. Enzyme amplification results from the coupling of catalytic processes, either by direct modification or by interaction with the product of the controlling enzyme.

U.S. Pat. No. 4,668,621 describes utilization of an enzyme-linked coagulation assay (ELCA) in an amplified immunoassay using a clotting cascade to enhance sensitivity. The process involves clot formation due to thrombin activated fibrin formation from soluble fibrinogen and labeled solubilized fibrinogen. Amplification of the amount of reportable ligand attached to solid-phase is obtained only by combining use of clotting factor conjugates with subsequent coagulation cascade reactions.

Substrate/cofactor cycling is another variation of enzyme-mediated amplification, and is based on the cycling of a cofactor or substrate that is generated by a primary enzyme label. The product of the primary enzyme is a catalytic activator of an amplifier cycle that responds in proportion to the concentration of substrate and hence the concentration of the enzyme label. An example of this type of substrate cycling system is described in U.S. Pat. No. 4,745,054.

Vary et al., Clin. Chem., 32, 1696 (1986) describes an enzyme amplification method suited to nucleic acid detection. This method is a strand displacement assay which uses the unique ability of a polynucleotide to act as a substrate label which can be released by a phosphorylase.

Bobrow et al., J. of Immunol. Methods, 125, 279 (1989) discloses a method to improve detection or quantitation of an analyte by catalyzed reporter deposition. Amplification of the detector signal is achieved by activating a conjugate consisting of a detectably labeled substrate specific for the enzyme system, wherein said conjugate then reacts with the analyte-dependent enzyme activation system to form an activated conjugate which deposits wherever receptor for the conjugate is immobilized.

Nucleotide hybridization assays have been developed as a means for detection of specific nucleic acid sequences. U.S. Pat. No. 4,882,269 discloses an amplified nucleic acid hybridization assay in which a target nucleic acid is contacted with a complementary primary probe having a polymeric tail. A plurality of second signal-generating probes capable of binding to the polymeric tail are added to achieve amplified detection of the target nucleic acid. Variations of this methodology are disclosed in PCT Application WO 89/03891 and European Patent Application 204510, which describe hybridization assays in which amplifier or multimer oligonucleotides are hybridized to a single-stranded nucleic acid unit which has been bound to the targeted nucleic acid segment. Signal amplification is accomplished by hybridizing signal-emitting nucleic acid bases to these amplifier and multimer strands. In all of these disclosures amplification is achieved by mechanisms which immobilize additional sites for attachment of signal-emitting probes.

Journal of Clinical Microbiol. 28,1968 (1990) describes a system for detection of amplified Chlamydia trachomatis DNA from cervical specimens by fluorometric quantitation in an enzyme immunoassay format which includes a polymerase chain reaction.

U.S. Pat. No. 5,665,539 describes a novel system and method for sensitive analyte detection using immuno-PCR. This consists of a biotinylated DNA which binds to analyte-dependent reporter-complex via a protein A-streptavidin chimeric protein. A segment of the DNA label is amplified by polymerase chain reaction and the products are detected by agarose gel electrophoresis.

In WO 9315229, Applicants disclose a method for the detection of an analyte through the formation of a complex comprising an analyte bound to a reporter having a nucleic acid label attached. Detection of the analyte is effected through amplification of the nucleic acid label.

It is an objective of the art to increase the sensitivity of analyte detection through the use of various novel signal generating reporter conjugates and amplification strategies. However, non-specific binding-signal due to non-selective binding of reporter conjugates to walls of the reaction tubes or to solid-phase reagents used in the assays even in the absence of analyte, is a serious problem in immunoassays. Non-specific binding signal thus diminishes the ratio of the analyte specific binding to analyte non-specific binding. This reduces the sensitivity of the detection limit for an analyte. The art has identified many factors that contribute to non-specific binding such as, protein-protein interaction, adsorptive surface of the solid-phase, Vogt et al., J. of Immunological Methods, 101, 43 (1987), the assay milieu and the efficiency of the wash solution.

To try and resolve this problem, a number of approaches have been used in this art by Vogt et al., J. of Immunological Methods, 101, 43 (1987), Graves, J. of Immunological Methods, 111, 167, (1988), Wedege et al., J. of Immunological Methods, 88, 233, (1986), Bodmer et al., J. of Immunoassay, 11,139, (1990), Pruslin et al., J. of Immunological Methods, 137, 27, (1991), Balde et al., J. of Biochem. and Biophys. Methods, 12, 271, (1986), Hauri et al., Analytical Biochemistry, 159, 386 (1986), Rodda et al., Immunological Investigations, 23, 421, (1994), Tovey et al., Electrophoresis, 10, 243, (1989), Kenney et al., Israel Journal Of Medical Sciences, 23, 732, (1987), Hashida et al., Analytical Letters, 18, 1143, (1985), Ruan et al., Ann Clin Biochem, 23, 54, (1985). To saturate the adsorptive surface, these investigators have used blocking agents such as, proteins bovine serum albumin (BSA), gelatin, casein, non-fat dry milk, polymers (poly vinyl alcohol) detergents (Tween 20), modified antibodies (Fab' and F(ab')₂), and combinations of blocking agents (BSA, Tween 20) and pentane sulfonate. These proteins have been chosen largely by convenience and empirical testing in ELISA systems, Vogt et al., J. of Immunological Methods, 101, 43 (1987).

Despite the numerous attempts in this art to use these approaches either individually or in combination, non-specific binding has not been eliminated. Therefore, increased assay detection sensitivity has been limited. Thus, there is a continuing, unmet need for a means to reduce assay background response and to improve the signal to noise ratio of binding assays. Further, approaches that leverage pre-existing infrastructure and reagents already present in modern molecular biology laboratories can provide reduced capital expenditures and hence provide economic advantages to the research community.

### SUMMARY

The present teachings provide a method of quantifying an analyte comprising; forming a reaction composition comprising a first proximity probe, a second proximity probe, and an analyte wherein the first proximity probe comprises a first binding moiety and a first coupled nucleic acid, and wherein the second proximity probe comprises a second binding moiety and a second coupled nucleic acid;binding the two proximity probes to two binding sites on the analyte, thereby forming a bound complex; interacting the first coupled nucleic and the second coupled nucleic acid of the bound complex with each other if they are in close proximity to each other, wherein said interacting comprises a hybridization reaction involving the coupled nucleic acids; cleaving the hybridized coupled nucleic acids to form cleaved nucleic acids, wherein the cleaving comprises a restriction endonuclease; quantitating at least one of the cleaved nucleic acids in a PCR, wherein the PCR comprises hybridizing a stem-loop primer to the at least one of the cleaved nucleic acids, wherein the stem-loop primer comprises a loop, self-complementary stem, and a 3′ cleaved nucleic acid portion, wherein the 3′ cleaved nucleic acid portion is complementary with the at least one cleaved nucleic acid, extending the stem-loop primer to form an extension reaction product; amplifying the extension reaction product to form an amplification product; and, quantitating the analyte. Additional methods, compositions, and kits are also provided.

### BRIEF DESCRIPTION OF THE DRAWINGS

The skilled artisan will understand that the drawings, described below, are for illustration purposes only. The drawings are not intended to limit the scope of the present teachings in any way.

FIG. 1 depicts certain aspects of various embodiments of the present teachings.

FIG. 2 depicts certain aspects of various embodiments of the present teachings.

FIG. 3 depicts certain aspects of various embodiments of the present teachings.

FIG. 4 depicts certain aspects of various embodiments of the present teachings.

FIG. 5 depicts certain aspects of various embodiments of the present teachings.

FIG. 6 depicts certain aspects of various embodiments of the present teachings.

FIG. 7 depicts certain aspects of various embodiments of the present teachings.

FIG. 8 depicts certain aspects of various embodiments of the present teachings.

FIG. 9 depicts certain aspects of various embodiments of the present teachings.

### SOME DEFINITIONS

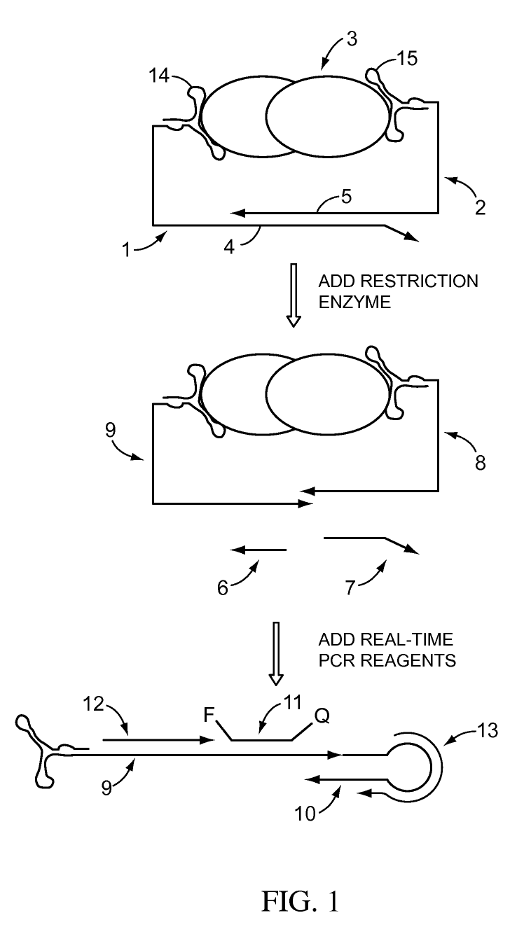

As used herein, the term “stem-loop primer” refers to a molecule comprising a 3′ target specific portion, a stem, and a loop. Illustrative stem-loop primers are depicted in FIG. 1, bottom (10), and are further described elsewhere in the application, as well as in U.S. patent application Ser. No. 10/947,460, Nucleic Acids Res. 2005 Nov. 27;33(20):e179, and Biochem Biophys Res Commun. 2006 Apr. 28;343(1):85-9. Epub 2006 Feb. 28. Depending on the context, a “3′ target-specific portion” can be referred to as a “3′ cleaved nucleic acid portion” or a “3′ truncated nucleic acid portion.” It will be appreciated that the stem-loop primers, as well as the other primers of the present teachings, can be comprised of ribonucleotides, deoxynucleotides, modified ribonucleotides, modified deoxyribonucleotides, modified phosphate-sugar-backbone oligonucleotides, nucleotide analogs, or combinations thereof. For some illustrative teachings of various nucleotide analogs etc, see Fasman, 1989, Practical Handbook of Biochemistry and Molecular Biology, pp. 385-394, CRC Press, Boca Raton, Fla., Loakes, N.A.R. 2001, vol 29:2437-2447, and Pellestor et al., Int J Mol Med. 2004 Apr.;13(4):521-5).

As used herein, the term “proximity probe” refers to a molecule that comprises a binding moiety and a coupled nucleic acid, as depicted in the various figures herein. Typically, the binding moieties correspond to sites on an analyte such as a protein. The present teachings also contemplate embodiments comprising what can be called ‘sandwich assays,’ in which for example a biotinylated antibody can bind a target analyte such as a protein, and proximity probes can comprise streptavidin and a coupled nucleic acid. Some such illustrative sandwich assays can be found described in U.S. Pat. No. 6,511,809 and U.S. Pat. No. 5,985,548, U.S. Pat. No. 5,665,539, and Published PCT Application WO 03/044231A1. In some embodiments, the proximity probes of the present teachings can comprise multivalent proximity probes, wherein each proximity probes comprises several binding moieties, as described for example in Published PCT Application WO 03/044231A1.

As used herein, the term “coupled nucleic acid” can refer to both a nucleic acid that is directly coupled to a proximity probe, as well as a nucleic acid that is coupled indirectly to a proximity probe, through for example any of a variety of linking moieties. The coupled nucleic acids of the present teachings, when present on proximity probes that interact with target analytes, can form double stranded structures that can be cleaved with a restriction enzyme.

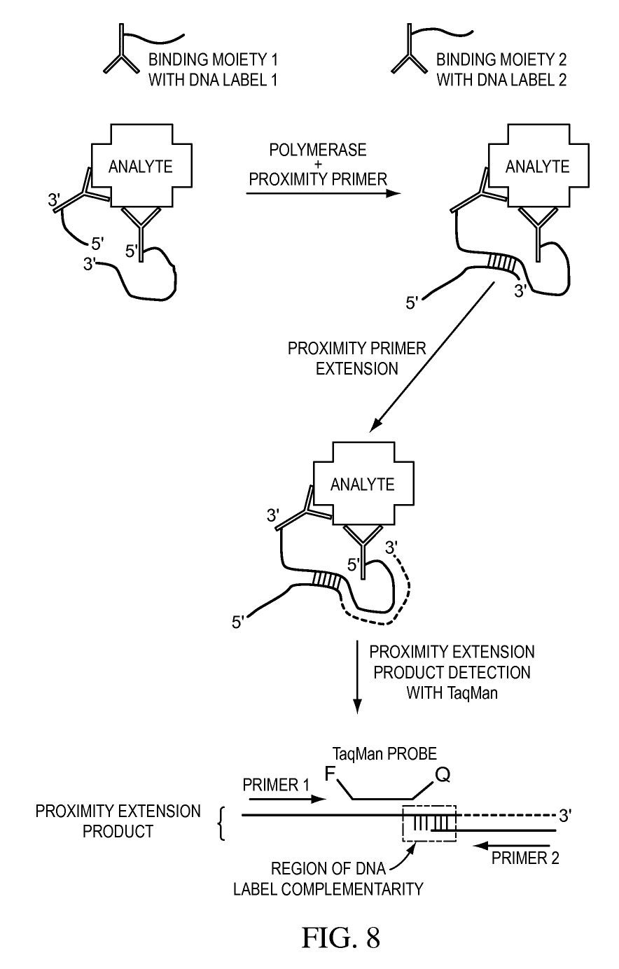

As used herein, the term “proximity primer” refers to a primer, as depicted for example in FIG. 8, which can hybridize to two coupled nucleic acids, thus serving as a splint. Upon hybridization, the proximity primer can be extended, and the resulting extension reaction product can be detected, thus allowing for detection of the target analyte.

As used herein, the terms “annealing” and “hybridization” are used interchangeably and mean the base-pairing interaction of one nucleic acid with another nucleic acid that results in formation of a duplex, triplex, or other higher-ordered structure. In certain embodiments, the primary interaction is base specific, e.g., A/T and G/C, by Watson/Crick and Hoogsteen-type hydrogen bonding. In certain embodiments, base-stacking and hydrophobic interactions may also contribute to duplex stability.

As used here, the term “detector probe” refers to a molecule used in an amplification reaction, typically for quantitative or real-time PCR analysis, as well as end-point analysis. Such detector probes can be used to monitor the amplification of the cleaved nucleic acid or the extended nucleic acid. In some embodiments, detector probes present in an amplification reaction are suitable for monitoring the amount of amplicon(s) produced as a function of time. Such detector probes include, but are not limited to, the 5′-exonuclease assay (TaqMan® probes described herein (see also U.S. Pat. No. 5,538,848) various stem-loop molecular beacons (see e.g., U.S. Pat. Nos. 6,103,476 and 5,925,517 and Tyagi and Kramer, 1996, Nature Biotechnology 14:303-308), stemless or linear beacons (see, e.g., WO 99/21881), PNA Molecular Beacons™ (see, e.g., U.S. Pat. Nos. 6,355,421 and 6,593,091), linear PNA beacons (see, e.g., Kubista et al., 2001, SPIE 4264:53-58), non-FRET probes (see, e.g., U.S. Pat. No. 6,150,097), Sunrise®/Amplifluor® probes (U.S. Pat. No. 6,548,250), stem-loop and duplex Scorpion™ probes (Solinas et al., 2001, Nucleic Acids Research 29:E96 and U.S. Pat. No. 6,589,743), bulge loop probes (U.S. Pat. No. 6,590,091), pseudo knot probes (U.S. Pat. No. 6,589,250), cyclicons (U.S. Pat. No. 6,383,752), MGB Eclipse ™ probe (Epoch Biosciences), hairpin probes (U.S. Pat. No. 6,596,490), peptide nucleic acid (PNA) light-up probes, self-assembled nanoparticle probes, and ferrocene-modified probes described, for example, in U.S. Pat. No. 6,485,901; Mhlanga et al., 2001, Methods 25:463-471; Whitcombe et al., 1999, Nature Biotechnology. 17:804-807; lsacsson et al., 2000, Molecular Cell Probes. 14:321-328; Svanvik et al., 2000, Anal Biochem. 281:26-35; Wolffs et al., 2001, Biotechniques 766:769-771; Tsourkas et al., 2002, Nucleic Acids Research. 30:4208-4215; Riccelli et al., 2002, Nucleic Acids Research 30:4088-4093; Zhang et al., 2002 Shanghai. 34:329-332; Maxwell et al., 2002, J. Am. Chem. Soc. 124:9606-9612; Broude et al., 2002, Trends Biotechnol. 20:249-56; Huang et al., 2002, Chem Res. Toxicol. 15:118-126; and Yu et al., 2001, J. Am. Chem. Soc 14:11155-11161. Detector probes can also comprise quenchers, including without limitation black hole quenchers (Biosearch), Iowa Black (IDT), QSY quencher (Molecular Probes), and Dabsyl and Dabcel sulfonate/carboxylate Quenchers (Epoch). Detector probes can also comprise two probes, wherein for example a fluor is on one probe, and a quencher is on the other probe, wherein hybridization of the two probes together on a target quenches the signal, or wherein hybridization on the target alters the signal signature via a change in fluorescence. Detector probes can also comprise sulfonate derivatives of fluorescenin dyes with SO3 instead of the carboxylate group, phosphoramidite forms of fluorescein, phosphoramidite forms of CY 5 (commercially available for example from Amersham). In some embodiments, interchelating labels are used such as ethidium bromide, SYBR® Green I (Molecular Probes), and PicoGreen® (Molecular Probes), thereby allowing visualization in real-time, or end point, of an amplification product in the absence of a detector probe. In some embodiments, real-time visualization can comprise both an intercalating detector probe and a sequence-based detector probe can be employed. In some embodiments, the detector probe is at least partially quenched when not hybridized to a complementary sequence in the amplification reaction, and is at least partially unquenched when hybridized to a complementary sequence in the amplification reaction. In some embodiments, probes can further comprise various modifications such as a minor groove binder (see for example U.S. Pat. No. 6,486,308) to further provide desirable thermodynamic characteristics. In some embodiments, detector probes can correspond to identifying portions or identifying portion complements.

### DETAILED DESCRIPTION OF EXEMPLARY EMBODIMENTS

Aspects of the present teachings may be further understood in light of the following exemplary embodiments, which should not be construed as limiting the scope of the present teachings in any way. The section headings used herein are for organizational purposes only and are not to be construed as limiting the described subject matter in any way. All literature and similar materials cited in this application, including but not limited to, patents, patent applications, articles, books, treatises, and internet web pages are expressly incorporated by reference in their entirety for any purpose. When definitions of terms in incorporated references appear to differ from the definitions provided in the present teachings, the definition provided in the present teachings shall control. It will be appreciated that there is an implied “about” prior to the temperatures, concentrations, times, etc discussed in the present teachings, such that slight and insubstantial deviations are within the scope of the present teachings herein. In this application, the use of the singular includes the plural unless specifically stated otherwise. Also, the use of “comprise”, “comprises”, “comprising”, “contain”, “contains”, “containing”, “include”, “includes”, and “including” are not intended to be limiting. It is to be understood that both the foregoing general description and the following detailed description are exemplary and explanatory only and are not restrictive of the invention.

As shown in FIG. 1, a first proximity probe (1) and a second proximity probe (2) bind an analyte (3, here a protein dimer, wherein the first proximity probe binds a binding site (14) on one member of the dimer and the second proximity probe binds a binding site (15) on the other member of the dimer). As a result of the binding of proximity probe one and proximity probe two to the analyte, proximity probe one's coupled nucleic acid (4) can form a complementary structure with proximity probe two's coupled nucleic acid (5). The resulting complementary structure can be cleaved with a restriction enzyme, thereby resulting in a variety of cleaved fragments (6, 7, 8, and 9). Hybridization of a stem-loop primer (10) to one of the cleaved fragments (here, 9) can result in the amplification and detection of the analyte, for example as with the depicted real-time PCR amplification comprising a TaqMan® probe (11), a forward primer (12), and a reverse primer (13). In some embodiments of the present teachings, the proximity probes 1 or 2 can be labeled, and the cleaved oligonucleotides, especially 6 or 7 in FIG. 1, bearing this label can be detected with a mobility dependent analysis technique such as capillary electrophoresis. Capillary electrophoresis is well known in the art, and is described for example in Jabeen et al., Electrophoresis. 2006 Jun.;27(12):2413-38

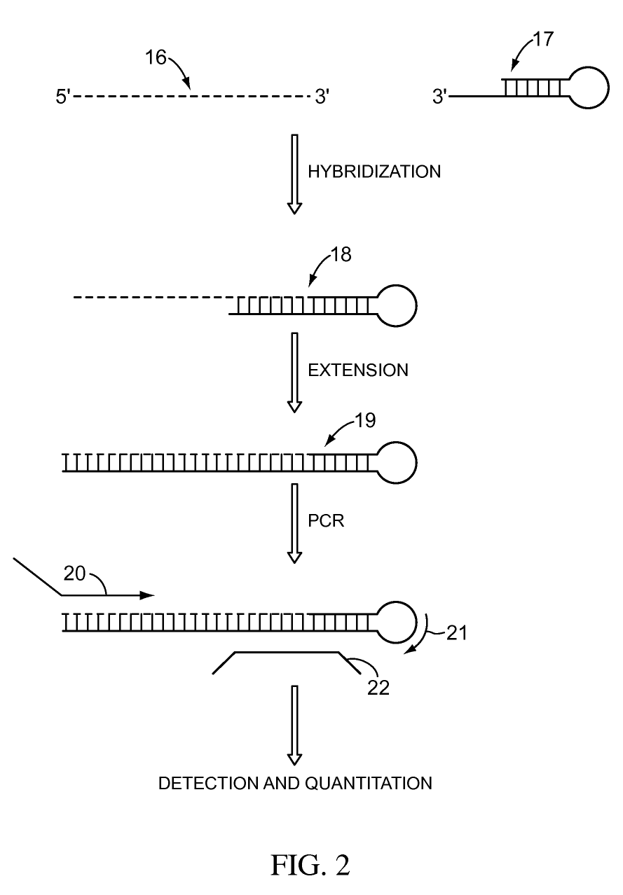

FIG. 2 depicts an exemplary TaqMan® reaction according to the present teachings employing a stem-loop primer. Here, a cleaved nucleic acid (16, dotted line) is illustrated to show the relationship with various components of the stem-loop primer (17), a detector probe (22), a reverse primer (21), and a forward primer (20), according to various non-limiting embodiments of the present teachings. For example, hybridization (18) of the stem-loop primer is followed by extension (19) and PCR. The PCR comprises a TaqMan® 5′ nuclease probe (22) a reverse primer (21) and a forward primer (20). Further illustrations of various PCR approaches using stem-loop primers can be found for example in U.S. Non-Provisional patent application Ser. No. 10/947,460, Nucleic Acids Res. 2005 Nov. 27;33(20):e179, and Biochem Biophys Res Commun. 2006 Apr. 28;343(1):85-9. Epub 2006 Feb. 28.

In some embodiments, the stem of the stem-loop primer comprises 12-16 nucleotides. In some embodiments, the 3′ cleaved nucleic acid portion, which hybridizes to the cleaved nucleic acid, comprises 5-8 nucleotides. In some embodiments, the loop of the stem-loop primer comprises 14-18 nucleotides. In some embodiments, the PCR comprises a real-time PCR amplification.

In some embodiments, the real-time PCR amplification comprises a 5′-nuclease cleavable probe, though it will be appreciated that any variety of real-time PCR approaches can be employed, incuding molecular beacons, PNA beacons, scorpion probes, etc. In some embodiments, the loop of the stem-loop primer comprises a universal reverse primer portion, such that when various analytes are queried with different proximity probes, and further analyzed in a PCR comprising a stem-loop primer, the loop remains the same such that the same reverse primer can always be employed in the PCR. Of course, the coupled nucleic acids can also be universal, as well as the resulting cleaved fragments, and the entire stem-loop primer, thus allowing for the economical and redundant use of universal PCR reagents across the large spectrum of analytes of interest. In some embodiments, the cleaved nucleic acid is 22 or fewer nucleotides in length. In some embodiments, the cleaved nucleic acid is 16 or fewer nucleotides in length. While the depicted emobidiments show shoreter cleaved nucleic acids, and their detection with stem-loop primers, it will be appreciated that longer cleaved nucleic acids are contemplated, and further that PCR amplification need not use a stem-loop primer, but can also employ more conventional linear primers. In some embodiments, the hybridization reaction involving the coupled nucleic acids comprises hybridization of the coupled nucleic acid from probe one with the coupled nucleic acid from probe two.

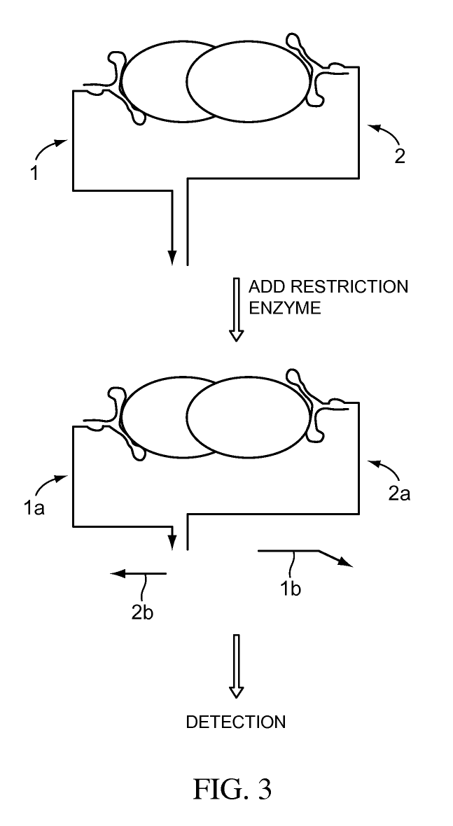

FIG. 3 depicts some embodiments of the present teachings. Here, an alternate configuration is depicted, wherein tailed aptamers comprising the coupled nucleic acid bind a homodimeric target protein. The bound first proximity probe (1) and the bound second proximity probe (2) form a complementary structure that can be cleaved with a restriction enzyme. The cleavage products include 2band 1b, as well as 1a and 2a. The 1a and 2a can remain hybridized to one another after the cleavage with the restriction enzyme.

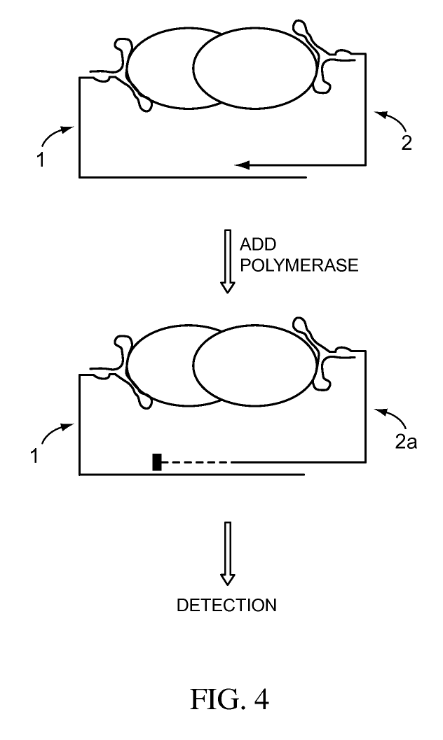

FIG. 4 depicts some embodiments of the present teachings. Here, tailed aptamers 1 and 2 bind a homodimeric target protein. Upon hybridization, the oligonucleotide coupled to the tailed aptamer 2 can be extended to a fixed point (shown as a rectangle) by omitting a single appropriately chosen dNTP from the reaction, thus forming 2a. Oligonucleotide 2a can then be specifically detected. For example, oligonucleotide 2a can be detected in a real-time PCR employing a stem-loop primer, as shown for example in FIG. 2.

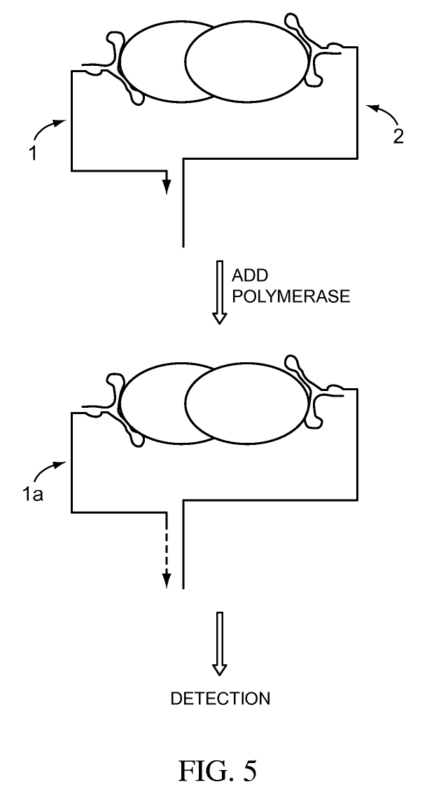

FIG. 5 depicts some embodiments of the present teachings. Here, tailed aptamers 1 and 2 bind a homodimeric target protein. Upon hybridization, the oligonucleotide coupled to the tailed aptamer 1 can be extended to a fixed point by adjusting the length of oligonucleotide 2. The product, 1a, can then be specifically detected. For example, oligonucleotide 1a can be detected in a real-time PCR employing a stem-loop primer, as shown for example in FIG. 2.

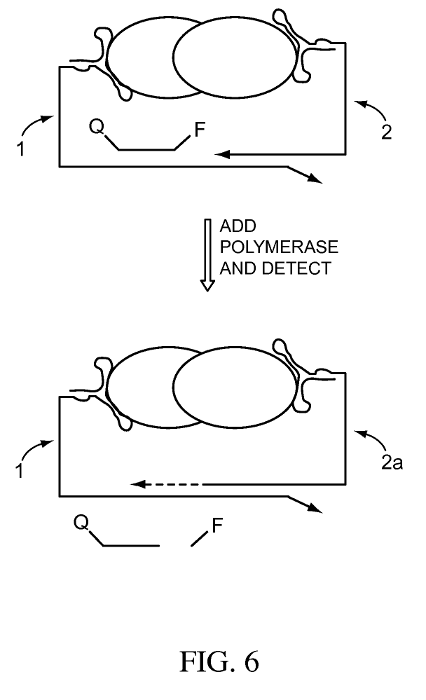

FIG. 6 depicts some embodiments of the present teachings. Here, tailed aptamers 1 and 2 bind a homodimeric target protein. Upon hybridization, the coupled nucleic acids provide a duplex structure to which a polymerase can bind and extend. Extension of the coupled oligonucleotide 2 can result in the cleavage of a 5′ nuclease probe (shown with a florophore (F) and a quencher (Q)) by the 5′-exonuclease activity of the polymerase, thus producing increased fluorescent signal.

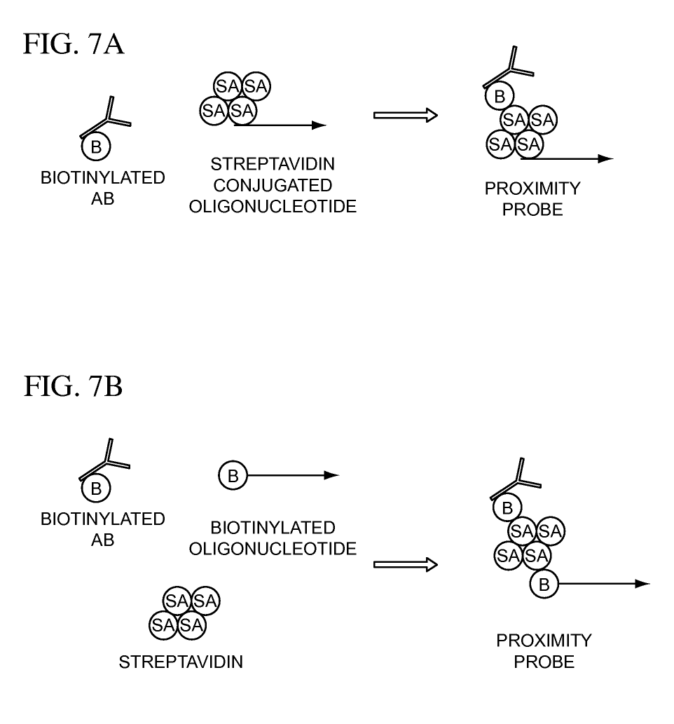

FIG. 7 depicts some embodiments of the present teachings. Here, different methods of constructing proximity probes are depicted. In one embodiment (top), a proximity probe is made by making a biotinylated antibody, and by making a streptavidinylated-conjugated oligonucleotide. Allowing for the high affinity interaction between the biotin and the streptavidin thus results in the formation of a proximity probe. In another embodiment (bottom), a proximity probe is made by making a biotinylated antibody, and by making a biotinylated oligonucleotide. Streptavidin can then be used to bridge the biotin on the antibody with the biotin on the oligonucleotide, thus forming a proximity probe.

FIG. 8 depicts some embodiments of the present teachings. Here, the DNA label two conjugated to binding moiety two does not contain enough complementarity, by itself, to the proximity primer to form a stable duplex. However, with the added stability contributed by DNA label one when it is brought into proximity with DNA label two by binding of both proximity probes to the analyte, the proximity primer can hybridize to both DNA label one and DNA label two, thus forming a stable duplex structure that can be extended by a polymerase. The resulting extension product can be detected, for example using a PCR with a TaqMan® detector probe. Thus, in some embodiments, the hybridization reaction involving the coupled nucleic acids comprises hybridization of the coupled nucleic acid from probe one and the coupled nucleic acid from probe two to a splint oligonucleotide, such as in proximity primer extension depicted in FIG. 8. In some embodiments, the splint oligonucleotide comprises a tail, wherein the tail is not complementary to either the first proximity probe or the second proximity probe. In some embodiments, the hybridization reaction involves hybridization of the coupled nucleic acid from probe one and the coupled nucleic acid from probe two to form hybridized coupled nucleic acids, wherein the hybridized coupled nucleic acids have an extendable end, wherein the extendable end is extended by a polymerase, thereby generating a duplex that can be recognized by a restriction enzyme. In some embodiments, the first probe, the second probe, or both, comprise a blocking oligonucleotide, wherein the blocking oligonucleotide is hybridized to the coupled nucleic acid, but is displaced by the hybridization reaction involving the coupled nucleic acids, as shown previously in FIG. 6.

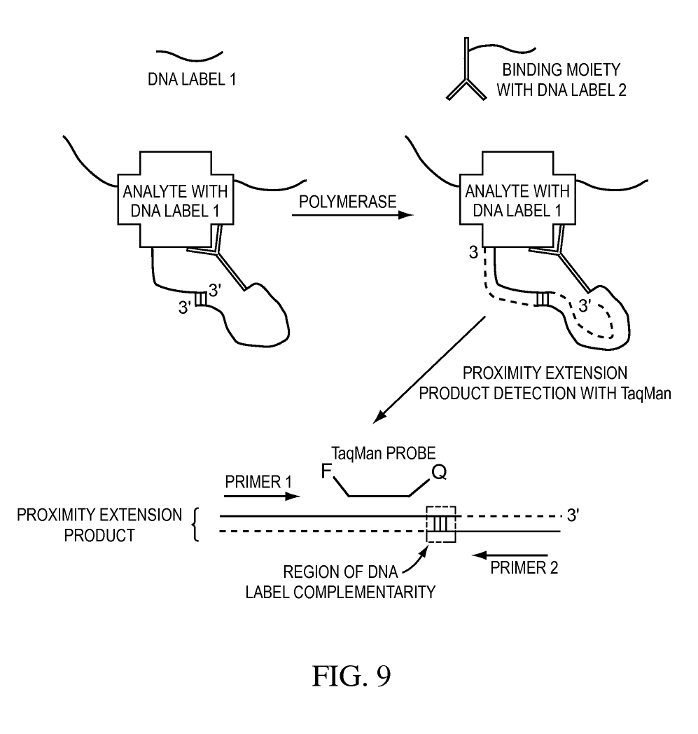

FIG. 9 depicts some embodiments of the present teachings. Here, the analyte to be queried is pre-labeled with a DNA label 1 (also referred to herein as an oligonucleotide label). This can be achieved by any suitable method, such as for example treating with biotin-NHS, which will covalently attach biotin to exposed amino groups from the N-termini and lysine side chains of proteins, followed by addition of streptavidin-linked DNA label 1. Once the DNA label 1-labeled sample is prepared, a binding moiety labeled with DNA label 2 is added, and proximity extension detection can be performed. Note that extension is similar to that of FIG. 8. In some embodiments the three DNA design of FIG. 8 can be employed. Without wishing to be limited by any particular theory, the embodiment depicted in FIG. 9 may suffer from cross reaction of the binding moiety to off-target proteins, which could still give a proximity extension detection signal if the off-target protein is also labeled with DNA label 1. Appropriate controls and routine experimentation should off-set this possible cross reaction. However, unlike FIG. 8, the embodiment of FIG. 9 can have only a single binding moiety, and therefore the sensitivity is dependent only on its properties.

In the embodiments depicted in FIGS. 8 and 9, each scheme employs two binding moieties, each binding to a separate portion of the analyte. Without being bound by particular theory, it is expected that the sensitivity of detection will be limited by the weaker of the two binding moieties. In some embodiments, it may be desirable, and the present teaching contemplate, and embodiment that uses only a single binding moiety. In some embodiments, the present teachings contemplate the use of double-stranded-dependent labels, for example Sybr Green. Double-stranded-dependent labels refers to a label that provides a detectably different signal value when it is exposed to double-stranded nucleic acid than when it is not exposed to double-stranded nucleic acid.

Thus, in some embodiments such double-stranded dependent labels can be employed to detect double stranded amplicons, for example double stranded PCR amplicons, resulting from amplification of a cleavage and/or extension product as produced by the interaction of two proximity probes. Examples include SYBR Green 1, Ethidium Bromide, Acridine Orange, and Hoechst 33258 (all available from Molecular Probes Inc., Eugene, Oreg.); TOTAB, TOED1 and TOED2 (Benson et al., Nucleic Acid Research, 21(24):5727-5735 (1993)); TOTO and YOYO (Benson et al., Analytical Biochemistry, 231:247-255 (1995). Exemplary double-stranded-dependent labels include, but are not limited to, certain minor groove binder dyes, including, but not limited to, 4′,6-diamino-2-phenylindole (Molecular Probes Inc., Eugene, Oreg.). Certain of the above-noted double-stranded-dependent labels and others are discussed, e.g., in Handbook of Fluorescent Probes and Research Chemicals, Sixth Edition, by Richard Haugland, Molecular Probes, Inc., Eugene, Oreg. (1996) (See, e.g., pages 149 to 151. Certain exemplary double-stranded-dependent labels are described, for example, in U.S. Pat. Nos. 5,994,056 and 6,171,785.

Universal Proximity-probes

When detecting an analyte the proximity-probes need not always bind to the analyte itself, but can instead bind via a first affinity reagent. In the case of an analyte with two binding sites, the first affinity reagents bind the analyte and the proximity-probes bind to these primary reagents. This strategy has advantages when designing universal proximity-probes useful with a plurality of different analytes. The laborious conjugation of nucleic acid sequences to various antibodies or other binding moieties can be overcome by making universal proximity-probes. These would comprise a secondary pair of binding moieties, each one capable of binding once to the Fc region (constant region) of a primary binding antibody pair. The Fc region is constant for many different antibodies of various specificities. So, the nucleic acids are conjugated to these secondary binding moieties, and used universally for the detection of many different analytes. The primary antibody pair is incubated with the analyte and the secondary reactive binding reagents are added and allowed to preferentially react when in a high local concentration. Such approaches are shown in FIG. 7 using streptavidin and biotin.

Competitive Proximity-probing for Analytes with Only One Binding Site

It is not always the case that two binding moieties are available for an analyte. This can be overcome by using a competitive assay. Herein, a purified amount of the analyte itself is conjugated to a nucleic acid and the one existing binding moiety is conjugated with the other reactive nucleic acid. When these two conjugates are permitted to react in a sample mixture containing an unknown amount of the analyte, the non-conjugated analyte of unknown amount in the sample will compete for binding to the binding moiety of the proximity-probe thereby decreasing the probability of the conjugated nucleic acids reacting. The signal from the reaction is in this case inversely proportional to the analyte concentration.

Multiplex Protein Detection Assays

Several analytes may be simultaneously detected by using several proximity-probe pairs, each specific for their distinct analyte. These proximity-probe pairs have unique nucleic acid sequences in order to distinguish them from other pairs. In one embodiment, the oligonucleotides all have the same PCR primer sites and the same restriction enzyme site but have unique identifier sequences. During the PCR the different amplicons representing the existence of different proteins are simultaneously amplified. These different PCR products may be detected by any of several methods, such as DNA microarrays, mass spectrometry, gel electrophoresis (different lengths of products), as well as stem-loop primer mediated PCR amplification, and various approaches for lower-plex decoding of multiplexed reactions, for example as discussed in U.S. patent application Ser. No. 10/693,609.

In some embodiments, a multiplexed pre-amplification can be performed, and single-plex PCR amplification reactions performed to detect and quantify one or more analytes, see for example Xtrana U.S. Pat. No. 6,605,451.

Screening Ligand Candidates in a Large Pool

Ligands to for example cell surface receptors can be found by screening cDNA expression clones for affinity towards said receptor. Such screening is usually carried out in various of solid phase formats where the known receptor is immobilised. Restriction digestion-mediated proximity-probing provides an alternative means to screen large sets of ligand candidates without the need for a solid phase. One needs an antibody capable of binding to the known receptor in such a way that it blocks binding by the unknown receptor ligand. To the receptor an oligonucleotide is conjugated, that is capable of hybridizing to a second oligonucleotide conjugated to the antibody, thereby allowing the hybridized oligonucleotides to be cut with a restriction enzyme. To a set of sample mixtures, the receptor and antibody is added to interact with a potential receptor ligand. The restriction digestion mix is added to the sample, and if a receptor ligand exists in the sample, cutting of the oligonucleotides will be inefficient due to the lack of nearness between them since receptor-antibody complexes fail to form in the prescence of the receptor ligand. A sample containing a potential ligand will therefore give a smaller signal. This method is not limited to receptors and their ligands, but could be used for all types of biomolecular interactions of interest.

Screening Drug Candidates from Large Libraries

In a fashion similar to the one described for the unknown ligand screening method one can also screen for drug candidates. For example a receptor and its ligand are both conjugated with oligonucleotides. In a mixture containing a competitive drug candidate the restriction digestion between the oligonucleotides will be inhibited since receptor ligand complexes fail to form. Large drug candidate libraries can thus be screened with minimal material use of receptor and its ligand.

Detection of Infectious Agents

By using probes with specificity for a surface molecule of an infectious agent such as a virus or an antibody, restriction digestion-mediated proximity probing could be used detect such agents at very low amounts. The two probes may be designed to bind to the same target if these are abundant on the surface and clustered near each other. The two probes may also bind to two different targets on the agent but also with the need to be near each other.

Using a Dimerising Affinity Moieity

If only one binding moiety can be constructed into a proximity-probe a multimeric affinity reagent can create proximity by dimerising the analytes, enabling their detection. This can be exemplified by an aptamer based binding moiety constructed into a proximity-probe and an antibody which dimerises the analyte. Many selex derived aptamers bind to only one site on the protein target. Since proximity probing requires the binding of at least two probes to each target in order to enable detection, these monovalent targets will be more difficult to detect. By adding to the incubation mixture a bivalent antibody (or other affinity reagent) capable of simultaneously binding two targets this may be overcome. The antibody must bind at a site separate from the selex aptamer so a complex of five molecules may form consisting of the antibody, two target proteins, and the two restriction digestable selex aptamer based proximity-probes.

In the presence of target, ligation of the aptamers is promoted by their proximity provided by the dimerising antibody. This system may alternatively be used to detect and quantify the antibody itself, by using constant amounts of the target and Selex aptamer.

Screening for Ligand-receptor Interaction Antagonists

When searching for antagonists of a ligand-receptor interaction for pharmaceutical use a sensitive, specific and rapid testing system is beneficial in order to screen vast libraries of candidate compounds. This is sometimes referred to as high throughput screening. The following is an example that shows how the present teachings can be designed to test whether or not a compound binds a certain receptor. This screening principle is here exemplified by PDGF-BB and its receptor interaction. By adding a surplus of soluble receptor to an incubation mix of PDGF-BB and proximity-probes, the binding of the probes to pdgf is blocked by the receptor and no signal is generated.

However, if a molecule which binds to the receptor in a competitive fashion is added to the incubation mix the PDGF is “liberated” and accessible to the proximtiy probes generating a signal.

In order to test this principle PDGF-AA can be used to mimic the action of an antagonist since it is capable of binding the pdgf-alfa receptor but not the aptamers. 6.4 pM PDGF-BB can be incubated with 5 pM of aptamer based proximity-probes and 2.5 nM of soluble PDGF-alpha receptor (in surplus). Upon addition of 100 nM PDGF-AA, which can bind the receptor but not the aptamers, a 3-fold increase in signal can be generated from the “liberated” PDGF-BB now accessible to the proximity-probes. The resulting signal resulted from a cleaved fragment in a PCR comprising stem-loop primers according to the present teachings can be used to infer information regarding antagonists of a ligand-receptor.

One of skill in the art, in light of the present teachings, will be able to employ routine experimentation to design a variety of reaction components for detecting and quantitating target analytes. For example, PCR primer and detector probe design is routine, as is the choice of restriction enzyme and restriction enzyme recognition sites. Illustrative teachings of these and related approaches can be found for example in Sambrook et al., Molecular Cloning, 3^{rd} Edition.

Kits

In certain embodiments, the present teachings also provide kits designed to expedite performing certain methods. In some embodiments, kits serve to expedite the performance of the methods of interest by assembling two or more components used in carrying out the methods. In some embodiments, kits may contain components in pre-measured unit amounts to minimize the need for measurements by end-users. In some embodiments, kits may include instructions for performing one or more methods of the present teachings. In certain embodiments, the kit components are optimized to operate in conjunction with one another.

Thus, in some embodiments the present teachings provide a kit for detecting an analyte comprising two proximity probes and a stem-loop primer. In some embodiments, the kit further comprises a restriction endonuclease. In some embodiments, the kit further comprises reagents for performing PCR amplification, including for example, a primer pair, a detector probe such as a 5′ nuclease probe, and a polymerase.

While the present teachings have been described in terms of these exemplary embodiments, the skilled artisan will readily understand that numerous variations and modifications of these exemplary embodiments are possible without undue experimentation. All such variations and modifications are within the scope of the current teachings. Aspects of the present teachings may be further understood in light of the additional guidance for performing examples consistent with the present teachings that are in routine molecular biology can be found in such treatises as Sambrook and Russell, Molecular Cloning 3^{rd} Edition. Additional methods for detecting and quantifying nucleic acids, and small nucleic acids, can be found in U.S. patent application Ser. No. 10/947,460, to Chen et al., U.S. patent application Ser. No. 10/944,153, to Lao et al., and U.S. patent application Ser. No. 10/881,362 to Karger et al.

All of the foregoing cited references are expressly incorporated by reference. Recognizing the difficulty of ipsissima verba in multiple documents related to the complex technology of molecular biology, it will be appreciated that when deviances in the nature of a definition are encountered, the definitions provided in the instant application will control.

We claim:

1. A method of quantifying an analyte comprising;
forming a reaction composition comprising a first proximity probe, a second proximity probe, and an analyte wherein the first proximity probe comprises a first binding moiety and a first coupled nucleic acid, and wherein the second proximity probe comprises a second binding moiety and a second coupled nucleic acid;
binding the two proximity probes to two binding sites on the analyte, thereby forming a bound complex;
interacting the first coupled nucleic and the second coupled nucleic acid of the bound complex with each other if they are in close proximity to each other, wherein said interacting comprises a hybridization reaction involving the coupled nucleic acids;
cleaving the hybridized coupled nucleic acids to form cleaved nucleic acids, wherein the cleaving comprises a restriction endonuclease;
quantitating at least one of the cleaved nucleic acids in a PCR, wherein the PCR comprises hybridizing a stem-loop primer to the at least one of the cleaved nucleic acids, wherein the stem-loop primer comprises a loop, self-complementary stem, and a 3′ cleaved nucleic acid portion, wherein the 3′ cleaved nucleic acid portion is complementary with the at least one cleaved nucleic acid,
extending the stem-loop primer to form an extension reaction product;
amplifying the extension reaction product to form an amplification product; and,
quantitating the analyte.

2. The method according to claim 1 wherein the stem of the stem-loop primer comprises 12-16 nucleotides.

3. The method according to claim 1 wherein the 3′ cleaved nucleic acid portion comprises 5-8 nucleotides.

4. The method according to claim 1 wherein the loop of the stem-loop primer comprises 14-18 nucleotides.

5. The method according to claim 1 wherein the PCR comprises a real-time PCR amplification.

6. The method according to claim 5 wherein the real-time PCR amplification comprises a detector probe.

7. The method according to claim 6 wherein the real-time PCR amplification comprises a PNA beacon.

8. The method according to claim 6 wherein real-time PCR amplification comprises a 5′-nuclesase cleavable probe.

9. The method according to claim 1 wherein the at least one cleaved nucleic acid is 22 or fewer nucleotides in length.

10. The method according to claim 1 wherein the at least one cleaved nucleic acid is 16 or fewer nucleotides in length.

11. The method according to claim 1 wherein the hybridization reaction involving the coupled nucleic acids comprise hybridization of the coupled nucleic acid from probe one with the coupled nucleic acid from probe two.

12. The method according to claim 1 wherein the hybridization reaction involving the coupled nucleic acids comprises hybridization of the coupled nucleic acid from probe one and the coupled nucleic acid from probe two to a splint oligonucleotide.

13. The method according to claim 12 wherein the splint oligonucleotide comprises a tail, wherein the tail is not complementary to either the first proximity probe or the second proximity probe.

14. The method according to claim 1 wherein the hybridization reaction involving the coupled nucleic acids comprises hybridization of the coupled nucleic acid from probe one and the coupled nucleic acid from probe two to form hybridized coupled nucleic acids, wherein the hybridized coupled nucleic acids have an extendable end, wherein the extendable end is extended by a polymerase, thereby generating a duplex that can be recognized by a restriction enzyme.

15. The method according to claim 1 wherein at least one of the first probe, the second probe, or both, comprise a blocking oligonucleotide, wherein the blocking oligonucleotide is hybridized to the coupled nucleic acid, but is displaced by the hybridization reaction involving the coupled nucleic acids.

16. A method for quantitating an analyte comprising;
binding of two proximity probes to two binding sites on the analyte, wherein each proximity probe comprises a binding moiety and a coupled nucleic acid;
allowing the binding moieties to bind the analyte and allowing the nucleic acids to interact with each other if they are in close proximity to each other, wherein said interacting comprises hybridization of the coupled nucleic acids to form hybridized coupled nucleic acids;
performing an extension reaction, wherein the extension reaction lacks at least one nucleotide, thereby allowing cessation of extension to form a truncated nucleic acid;
hybridizing a primer to the truncated nucleic acid;
extending the primer to form an extension reaction product;
amplifying the extension reaction product to form an amplification product; and,
quantitating the analyte.

17. The method according to claim 16 wherein the amplifying comprises PCR.

18. The method according to claim 17 wherein the PCR comprises a real-time PCR amplification.

19. The method according to claim 18 wherein the real-time PCR amplification comprises a detector probe.

20. The method according to claim 19 wherein the real-time PCR amplification comprises a PNA beacon.

21. The method according to claim 19 wherein real-time PCR amplification comprises a 5′-nuclease cleavable probe.

22. A method for quantitating an analyte comprising;
binding of two proximity probes to a binding site on the analyte, wherein each proximity probe comprises a binding moiety and a coupled nucleic acid;
allowing the binding moiety to bind the analyte and allowing the nucleic acids to interact with each other if they are in close proximity to each other, wherein said interacting comprises a hybridization of the coupled nucleic acid from probe one and the coupled nucleic acid from probe two to a proximity primer;
performing an extension reaction, wherein the extension reaction comprises a extension of the proximity primer to form an extension product;
hybridizing a primer to the extension product;
extending the primer to form an extension reaction product;
amplifying the extension reaction product to form an amplification product; and,
quantitating the analyte.

23. The method according to claim 22 wherein the amplifying comprises PCR.

24. The method according to claim 23 wherein the PCR comprises a real-time PCR amplification.

25. The method according to claim 24 wherein the real-time PCR amplification comprises a detector probe.

26. The method according to claim 25 wherein the real-time PCR amplification comprises a PNA beacon.

27. The method according to claim 25 wherein real-time PCR amplification comprises a 5′-nuclease cleavable probe.

28. A method for quantitating an analyte comprising;
labeling an analyte with an oligonucleotide label;
binding a proximity probe to a binding site on the analyte, wherein the proximity probe comprises a binding moiety and a coupled nucleic acid;
allowing the oligonucleotide label to interact with the coupled nucleic acid, wherein said interacting comprises hybridization of the coupled nucleic acid from the proximity probe with the oligonucleotide label on the analyte;
performing an extension reaction, wherein the extension reaction comprises a extension of the coupled nucleic acid on the proximity probe, extension of the oligonucleotide label on the analyte, or extension of both of the coupled nucleic acid on the proximity probe and the oligonucleotide label on the analyte, to form at least one extension product;
hybridizing a primer to the extension product;
extending the primer to form an extension reaction product;
amplifying the extension reaction product to form an amplification product; and,
quantitating the analyte.

29. The method according to claim 28 wherein the amplifying comprises PCR.

30. The method according to claim 29 wherein the PCR comprises a real-time PCR amplification.

31. The method according to claim 30 wherein the real-time PCR amplification comprises a detector probe.

32. The method according to claim 30 wherein the real-time PCR amplification comprises a PNA beacon.

33. The method according to claim 30 wherein real-time PCR amplification comprises a 5′-nuclease cleavable probe.

35. A kit for detecting an analyte comprising two proximity probes and a stem-loop primer.

36. The kit according to claim 35 further comprising a restriction endonuclease.

37. The kit according to claim 35 further comprising reagents for performing a PCR amplification, including a primer pair, a detector probe, and a polymerase.

38. The kit according to claim 37 further comprising a proximity primer.

39. A composition comprising;
an analyte;
a first proximity probe;
a second proximity probe, wherein the first proximity probe comprises a nucleic acid conjugate that is hybridized to a nucleic acid conjugate of the second proximity probe; and,
a restriction endonuclease.

40. The composition according to claim 39 wherein the analyte is a protein.

41. The composition comprising;
an analyte;
a first proximity probe;
a second proximity probe, wherein the first proximity probe comprises a nucleic acid conjugate that is hybridized to a proximity primer, and wherein a nucleic acid conjugate of the second proximity probe is hybridized to the proximity primer; and,
a polymerase.

42. The composition according to claim 41 wherein the analyte is a protein.
